(19) United States
(12) Patent Application Publication
(10) Pub. No.: US 2011/0218433 A1
(43) Pub. Date: Sep. 8, 2011

(54) Biopsy Marker Delivery Device

(76) Inventors: Trevor W.V. Speeg, Williamsburg, OH (US); Julie A. Horning, Loveland, OH (US); Edward A. Rhad, Fairfield, OH (US); Jacob S. Gee, Cincinnati, OH (US); Tiffiny K. Aguon, Maineville, OH (US); Jessica P. Leimbach, Cincinnati, OH (US)

(21) Appl. No.: 12/787,492

(22) Filed: May 26, 2010

Related U.S. Application Data
(60) Provisional application No. 61/309,550, filed on Mar. 2, 2010.

(51) Int. Cl. A61B 10/02

(52) U.S. Cl. 600/432

(57) ABSTRACT

A biopsy site marker delivery device includes a relatively flexible marker deployer tube having a closed distal end, a push rod slidably disposed within the tube, and at least one marker disposed in the tube. The deployer tube includes a side exit port with an adjacent ramp for transversely dispensing the marker upon distal advancement of the push rod within the tube. An elongate member such as a sleeve is operable to guide and/or stabilize the flexible deployer tube as the tip of the tube is inserted into an access channel such as a channel provided by a cannula or biopsy device. An internal package member is configured to support the marker delivery device and the elongate member, with the elongate member being positioned over the side exit port to prevent the marker from inadvertently exiting the side exit port. The package member may reside in a sterile packaging envelope

### PRIORITY

This application claims priority to U.S. Provisional Patent Application Ser. No. 61/309,550, filed Mar. 2, 2010, entitled “Biopsy Marker Delivery Device,” the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. An exemplary biopsy device is the MAMMOTOME® brand device from Ethicon Endo-Surgery, Inc. of Cincinnati, Ohio. Biopsy devices may be used under stereotactic guidance, ultrasound guidance, MRI guidance, or otherwise.

Further exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2006/0074345, entitled “Biopsy Apparatus and Method,” published Apr. 6, 2006; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Pub. No. 2009/0171242, entitled “Clutch and Valving System for Tetherless Biopsy Device,” published Jul. 2, 2009; U.S. Non-Provisional patent application Ser. No. 12/335,578, entitled “Hand Actuated Tetherless Biopsy Device with Pistol Grip,” filed Dec. 16, 2008; U.S. Non-Provisional patent application Ser. No. 12/337,942, entitled “Biopsy Device with Central Thumbwheel,” filed Dec. 18, 2008; and U.S. Non-Provisional patent application Ser. No. 12/483,305, entitled “Tetherless Biopsy Device with Reusable Portion,” filed Jun. 12, 2009. The disclosure of each of the above-cited U.S. Patents, U.S. Patent Application Publications, and U.S. Patent Applications is incorporated by reference herein.

In some settings, it may be desirable to mark the location of a biopsy site for future reference. For instance, one or more markers may be deposited at a biopsy site before, during, or after a tissue sample is taken from the biopsy site. Exemplary marker deployment tools include the MAMMOMARK™, MICROMARK®, and CORMARK™ brand devices from Ethicon Endo-Surgery, Inc. of Cincinnati, Ohio. Further exemplary devices and methods for marking a biopsy site are disclosed in U.S. patent application Ser. No. 12/563,360, entitled “Flexible Biopsy Marker Delivery Device,” filed Sep. 21, 2009; U.S. patent application Ser. No. 12/564,315, entitled “Biopsy Marker Delivery Device,” filed Sep. 22, 2009; U.S. patent application Ser. No. 12/565,968, entitled “Biopsy Marker Delivery Device with Positioning Component,” filed Sep. 24, 2009; U.S. Pub. No. 2009/0209854, entitled “Biopsy Method,” published Aug. 20, 2009; U.S. Pub. No. 2009/0270725, entitled “Devices Useful in Imaging,” published Oct. 29, 2009; U.S. Pub. No. 2010/0049084, entitled “Biopsy Marker Delivery Device,” published Feb. 25, 2010; U.S. Pat. No. 6,228,055, entitled “Devices for Marking and Defining Particular Locations in Body Tissue,” issued May 8, 2001; U.S. Pat. No. 6,371,904, entitled “Subcutaneous Cavity Marking Device and Method,” issued Apr. 16, 2002; U.S. Pat. No. 6,993,375, entitled “Tissue Site Markers for In Vivo Imaging,” issued Jan. 31, 2006; U.S. Pat. No. 6,996,433, entitled “Imageable Biopsy Site Marker,” issued Feb. 7, 2006; U.S. Pat. No. 7,044,957, entitled “Devices for Defining and Marking Tissue,” issued May 16, 2006; U.S. Pat. No. 7,047,063, entitled “Tissue Site Markers for In Vivo Imaging,” issued May 16, 2006; U.S. Pat. No. 7,229,417, entitled “Methods for Marking a Biopsy Site,” issued Jun. 12, 2007; and U.S. Pat. No. 7,465,279, entitled “Marker Device and Method of Deploying a Cavity Marker Using a Surgical Biopsy Device,” issued Dec. 16, 2008. The disclosure of each of the above-cited U.S. Patents, U.S. Patent Application Publications, and U.S. Patent Applications is incorporated by reference herein.

It may be desirable in some (but not necessarily all) settings to deploy markers from a cannula type deployer into the biopsy site, such as with a flexible tubular deployer. It may also be desirable in some (but not necessarily all) settings to substantially prevent a marker from unintentionally falling out of a deployer, such as at time prior to the intended deployment. In addition, it may be desirable in some (but not necessarily all) settings to guide and/or steady the tip of a flexible tube of a deployer as the tip is inserted into a biopsy device or other access device used to provide a path to a biopsy site.

While several structures and methods have been made and used for providing and using a marker applier device, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary marker delivery device.

FIG. 2 depicts a cross-sectional view of a distal portion of the marker delivery device of FIG. 1.

FIG. 3 depicts a side view the marker delivery device of FIG. 1 inserted in a biopsy device needle, with the biopsy device needle shown in cross-section, and with a marker being deployed through aligned lateral openings of the marker delivery device and the biopsy device needle.

FIG. 4 depicts a perspective view of a combination of the marker delivery device of FIG. 1 and an exemplary outer packaging, with the marker delivery device being removed from the outer packaging by a single hand, and with the biopsy marker shown supported on an internal package member.

FIG. 5 depicts a perspective view of the marker delivery device of FIG. 1 with a conventional marker delivery device internal package member.

FIG. 6 depicts a perspective view of the marker delivery device of FIG. 1 with an exemplary guide sleeve and associated internal package member.

FIG. 7 depicts an enlarged perspective view of the distal end of the assembly of FIG. 6.

FIG. 8A depicts a perspective view of the marker delivery device of FIG. 1 and a sleeve in position to be inserted into an access cannula, the sleeve shown positioned to cover the side exit port of the deployer tube, and the distal end of the sleeve positioned against a proximal opening in the access cannula.

FIG. 8B depicts a perspective view of the assembly of FIG. 8A, with the deployer tube advanced distally relative to the access cannula so that the side exit port extends distally beyond the access cannula, and with the guide sleeve distal end maintained in position against the proximal opening of the access cannula, but with the sleeve disposed proximally relative to the deployer tube so that a proximal end of the sleeve is adjacent a grip portion of the marker delivery device.

FIG. 9 depicts a cross-sectional view of the marker delivery device of FIG. 8B, with the proximal end of the guide sleeve adjacent the grip portion of the marker delivery device.

FIG. 10 depicts a perspective view of the sleeve of the assembly of FIG. 8.

FIG. 11 depicts a perspective view of another exemplary sleeve.

FIG. 12 depicts a perspective view of yet another exemplary sleeve.

FIG. 13 depicts a perspective view of yet another exemplary sleeve.

FIG. 14 depicts a perspective view of the sleeve of FIG. 13 approaching the access cannula of FIGS. 8A-8B.

FIG. 15 depicts a perspective view of yet another exemplary sleeve.

FIG. 16 depicts a perspective view of an exemplary clip sleeve.

FIG. 17 depicts a perspective view of another exemplary clip sleeve, in an open configuration.

FIG. 18 depicts a perspective view of the clip sleeve of FIG. 17 in a closed configuration.

FIG. 19 depicts a perspective view of the marker delivery device of FIG. 1 with another exemplary guide sleeve and associated internal package member.

FIG. 20 depicts an enlarged perspective view of the distal end of the assembly of FIG. 19.

FIG. 21 depicts a perspective view of the marker delivery device of FIG. 1 with yet another exemplary guide sleeve and associated internal package member.

FIG. 22 depicts an enlarged perspective view of the distal end of the assembly of FIG. 21.

FIG. 23 is a cross-sectional schematic view of the distal end of FIG. 22, taken along line 23-23 of FIG. 22, showing the sleeve with projections extending into slots in first and second tabs.

FIG. 24 is a top view of the marker delivery device of FIG. 1 with yet another exemplary guide sleeve and associated internal package member.

FIG. 25 is a bottom view of the assembly of FIG. 24.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

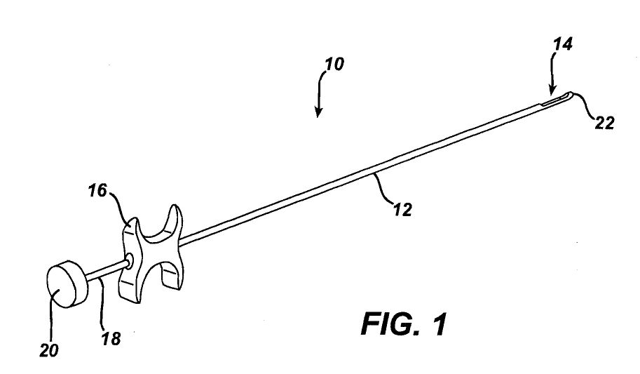

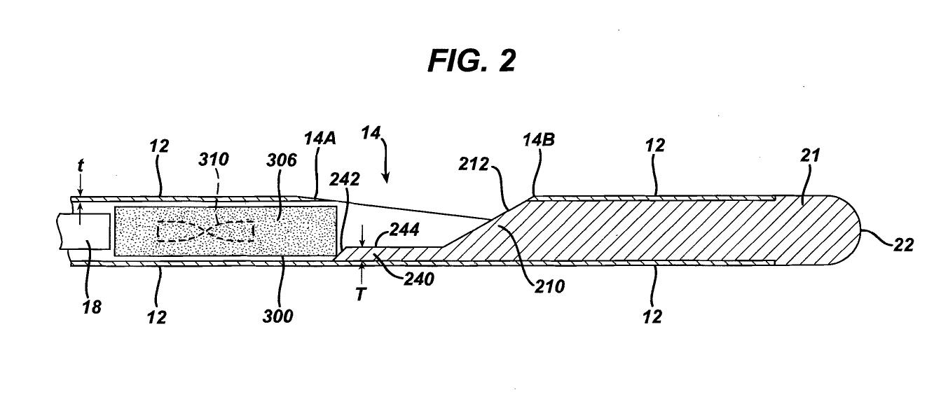

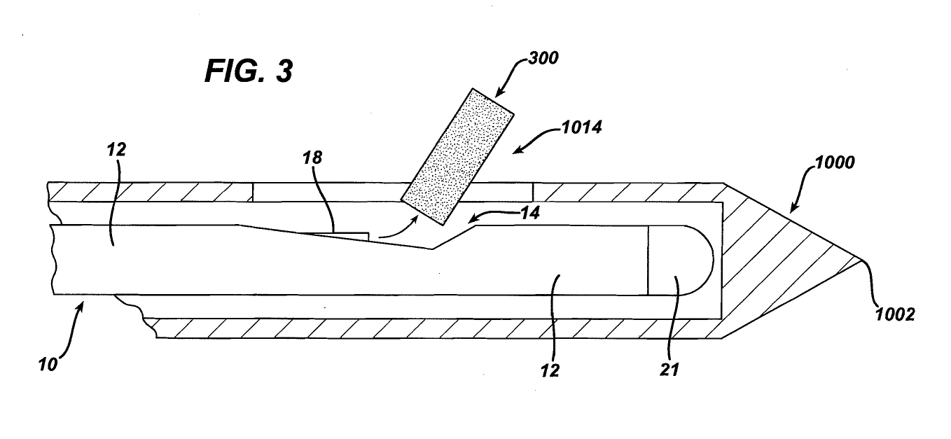

FIGS. 1-3 illustrate an exemplary marker delivery device (10), which includes an elongate flexible outer deployer tube (12) a side opening (14) formed near to, but spaced proximally from, the distal end of the deployer tube (12). A grip (16) is provided at the proximal end of deployer tube (12). A push rod (18) extends coaxially in deployer tube (12) such that push rod (18) is configured to translate within deployer tube (12) to displace one or more markers (300) through the side opening (14) as shown in FIGS. 2-3. Deployer tube (12) and rod (18) may be relatively flexible in bending so that the deployer tube (12) may be inserted along a straight or curved path to deploy a marker element (300) at a biopsy site. A plunger (20) is provided at the proximal end of rod (18) for pushing rod (18) distally in deployer tube (12) to deploy a marker (300) out of the deployer tube (12). A user may grasp grip (16) with two fingers, and may push on plunger (20) using the thumb on the same hand, so that marker delivery device (10) may be operated by a user's single hand. A spring (19) or other feature may be provided about rod (18) to bias rod (18) proximally relative to grip (16) and deployer tube (12).

FIG. 2 depicts a cross-sectional view of a distal portion of marker delivery device (10). FIG. 2 shows a biopsy marker (300) disposed in the internal lumen (15) of deployer tube (12). Marker (300) of the present example comprises a biodegradable or otherwise bioresorbable body (306), such as a generally cylindrically shaped body of collagen or other suitable polymeric material, and a metallic, generally radiopaque marker element (310) (shown in phantom) disposed within or otherwise carried by the body (306). Marker (300) may be composed and/or configured in accordance with the teachings of any of the various U.S. Patents, U.S. Patent Application Publications, or U.S. Patent Applications cited herein. Alternatively, marker (300) may have any other suitable composition and/or configuration. It should also be understood that a plurality of markers (300) may be provided within deployer tube (12) (e.g., in an end-to-end arrangement, etc.), if desired. If a plurality of markers (300) are used, it should be understood that a plurality of markers (300) within a single deployer tube (12) may have the same size, shape, and/or composition. Alternatively, a plurality of markers (300) within a single deployer tube (12) may have different sizes, shapes, and/or compositions.

Deployer tube (12) may be formed of any suitable metallic or non-metallic material, or even a combination of metallic and non-metallic materials. In the present example, deployer tube (12) is formed of a relatively flexible, thin walled hollow tube formed of a suitable medical grade plastic or polymer. One suitable material is a thermoplastic elastomer, such as Polyether block amide (PEBA), such as is known under the tradename PEBAX. Deployer tube (12) may thus be formed of PEBAX, and may be substantially transparent to visible light and X-ray. Side opening (14) may be formed by cutting away a portion of the wall of deployer tube (12); or using any other suitable technique. Side opening (14) communicates with an internal lumen (15) of deployer tube (12). Side opening (14) extends axially (in a direction parallel to the axis of lumen (15)) from a proximal opening end (14A) to a distal opening end (14B), as illustrated in FIG. 2.

The distal tip (22) extending from the distal end of deployer tube (12) may be rounded as shown in FIG. 2. Of course, distal tip (22) may alternatively have any other suitable configuration. Still referring to FIG. 2, marker delivery device (10) of the present example has the distal end of deployer tube (12) closed by a unitary endpiece (21) formed in place in the distal end of deployer tube (12), with a part of endpiece (21) extending into internal lumen (15) of deployer tube (12). Distal endpiece (21) may be a molded or cast component, and may provide an integrally formed combination of tip (22), a ramp (210) having a ramp surface (212), and a marker engaging element (240). Ramp surface (212) may aid in directing marker (300) from internal lumen (15) through side opening (14). Marker engaging element (240) may be employed to substantially retain marker (300) in internal lumen (15) until the user intends to deploy marker (300).

Marker engaging element (240) of the present example is disposed within internal lumen (15), and at least a portion of marker engaging element (240) is disposed distally of proximal end (14A) of side opening (14). Marker engaging element (240) extends along a portion of the floor of lumen (15) under the opening (14); and is positioned to reinforce the portion of deployer tube (12) in which opening (14) is formed. For instance, by positioning marker engaging element (240) underneath opening (14) as shown in FIG. 2, marker engaging element (240) may help to substantially stiffen deployer tube (12) in the region where the wall of deployer tube (12) is cut to form opening (14). Marker engaging element (240) extends from the proximal-most portion of ramp surface (212), and does not extend proximally of side opening (14), though in some other versions, a portion of marker engaging element (240) could extend proximally of opening (14) if desired. Marker engaging element (240) is in the form of a step having a generally uniform thickness (T) along the element's axial length, except that marker engaging element (240) has a tapered proximal end (242) in the present example. Tapered proximal end (242) may form an included angle with the longitudinal axis of the lumen (15) (included angle with a horizontal line in FIG. 2) of about 45 degrees, while ramp surface (212) may form an included angle with the longitudinal axis of about 30 degrees. Of course, these angles are mere examples, and it should be understood that any other suitable angles may be used. As shown in FIG. 2, an upwardly facing surface (244) (surface facing opening (14)) of marker engaging element (240) extends distally from tapered proximal end (242) of marker engaging element (240) to contact ramp surface (212).

The thickness (T) of marker engaging element (240) may be greater than the wall thickness (t) of deployer tube (12). For instance, in some versions, thickness (T) is at least about twice the thickness (t). By way of example only, the thickness (T) of marker engaging element (240) may be between about 0.018 inch to about 0.040 inch; and the wall thickness (t) of deployer tube (12) may be between about 0.005 inch to about 0.008 inch. The internal diameter of lumen (15) may be about 0.120 inch. Of course, any other suitable dimensions may be used for these components. It should be understood that, as with other components described herein, marker engaging element (240) may have any other suitable configuration, and may even be omitted as desired.

If desired, the marker engaging element (240), ramp (210), and/or tip (22) may be formed of, or include, a material that is relatively more radiopaque than the wall of deployer tube (12). For instance, where marker engaging element (240), ramp (210), and tip (22) are formed as an integral endpiece (21), endpiece (21) may include a radiopaque additive, such as barium sulfate. My way of example only, endpiece (21) may be a component molded of PEBAX, with about 20 percent by weight barium sulfate added to the molten PEBAX mold composition. The relatively more radiopaque marker engaging element (240), ramp (210), and tip (22) may be useful in distinguishing the position of those components using radiographic imaging. Also, where ramp (210) and/or step of marker engaging element (240) is/are positioned in association with opening (14), the addition of a radiopaque material may help identify the position of opening (14), and the position of the marker (300) relative to opening (14), before, during, or after deployment of marker (300).

In some versions, deployer tube (12) is generally transparent to visible light and x-ray; while endpiece (21) is generally opaque to visible light and x-ray. If desired, endpiece (21) may be colored with a dye or other suitable colorant in a liquid mold composition. For example, it may be desirable to have different size markers (e.g. length and/or diameter, etc.) for different biopsy procedures. For instance, it may be desirable to provide a larger marker if a relatively large biopsy sample is taken; and a smaller marker if a relatively small biopsy sample is taken. Endpiece (21) may be colored using one of multiple colors to indicate the size of the marker disposed in deployer tube (12). For instance, if three marker sizes are provided, endpiece (21) may be colored one of three colors to identify which of the marker sizes are disposed in the particular marker delivery device (10). Endpiece (21) may also be colored to indicate a particular size (e.g., diameter or length, etc.) or type of biopsy needle with which the marker delivery device (10) is to be used. Additionally, multiple marker delivery devices (10) may be packaged in kit form, with the kit including marker delivery devices (10) having different size markers and correspondingly colored endpieces (21). Still other variations will be apparent to those of ordinary skill in the art in view of the teachings herein.

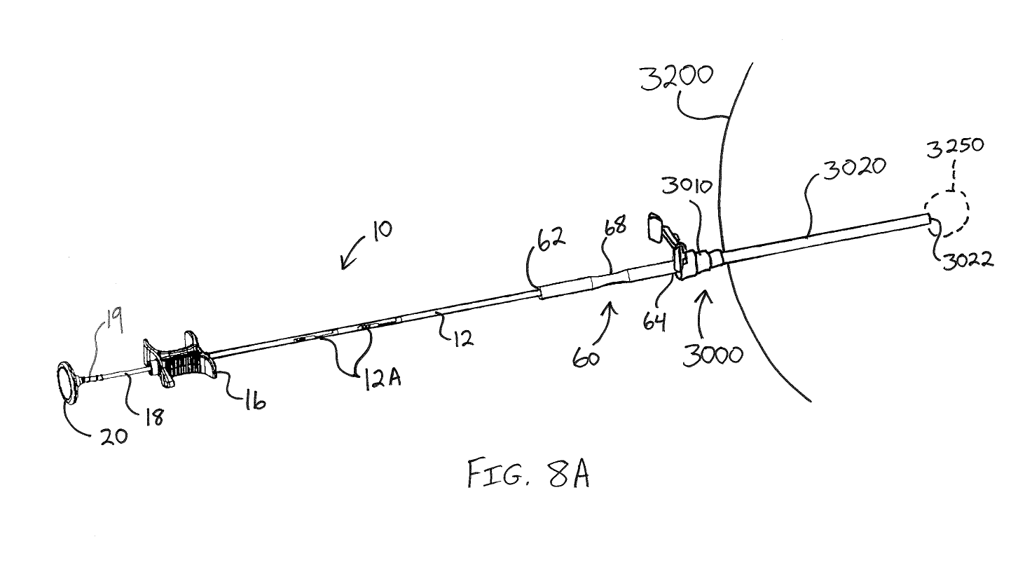

As shown in FIG. 3, marker delivery device (10) may be used to deploy a marker (300) to mark a particular location within a patient. For instance, FIG. 3 depicts marker delivery device (10) being used through a biopsy needle (1000). Needle (1000) of this example has a closed distal end with a piercing tip (1002); and a lateral tissue receiving aperture (1014). Marker delivery device (10) may be introduced to a biopsy site through biopsy needle (1000), which can be the same needle (1000) used to collect a tissue sample from the biopsy site. Biopsy needle (1000) may be of the type used with single insertion, multiple sample vacuum assisted biopsy devices. Several such biopsy devices are disclosed in the various U.S. Patents, U.S. Patent Application Publications, and U.S. Patent Applications that have been referred to and incorporated by reference herein, though it should be understood that marker delivery device (10) may be used with various other biopsy devices. Alternatively, marker delivery device may be used with an access cannula, as described in more detail below with reference to FIGS. 8A-8B.

FIG. 3 shows the distal end of marker delivery device (10) disposed within the interior of needle (1000). It should be understood that needle (1000) may be positioned in tissue, and a biopsy sample may be obtained through opening (1014), thereby providing a biopsy cavity adjacent opening (1014). Then, after the tissue sample has been obtained and transferred proximally through needle (100), and without removing needle (1000) from the patient's tissue, marker delivery device (10) may be inserted through a proximal opening (not shown) in needle (1000). In particular, needle (1000) and marker delivery device (10) are positioned such that opening (14) of deployer tube (12) and opening (1014) of needle (1000) are substantially aligned axially and circumferentially. Then, with marker delivery device (10) and needle (1000) so positioned at the biopsy site, push rod (18) may be advanced to deploy marker (300) up ramp surface (212), through opening (14), and then through opening (1014), into the biopsy cavity.

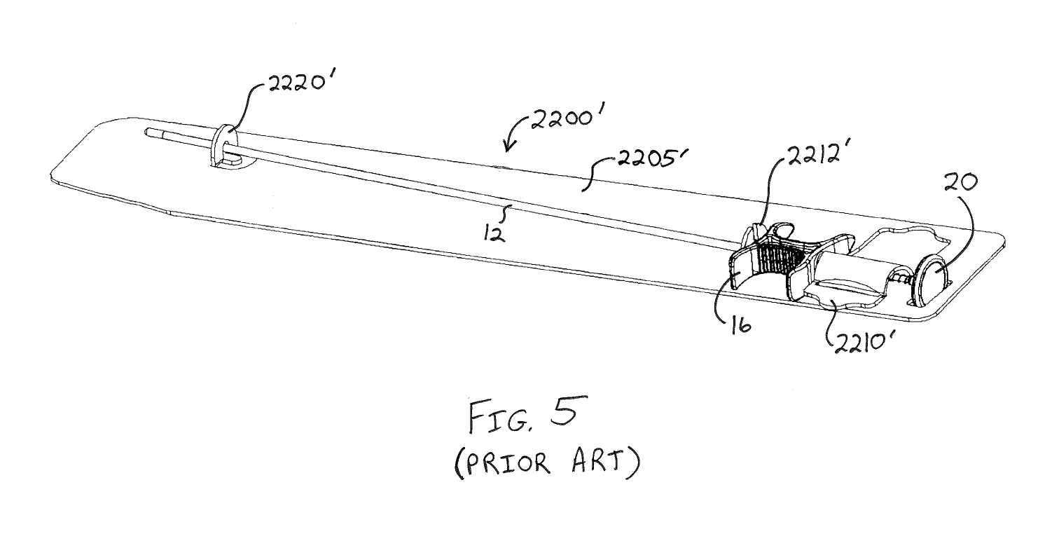

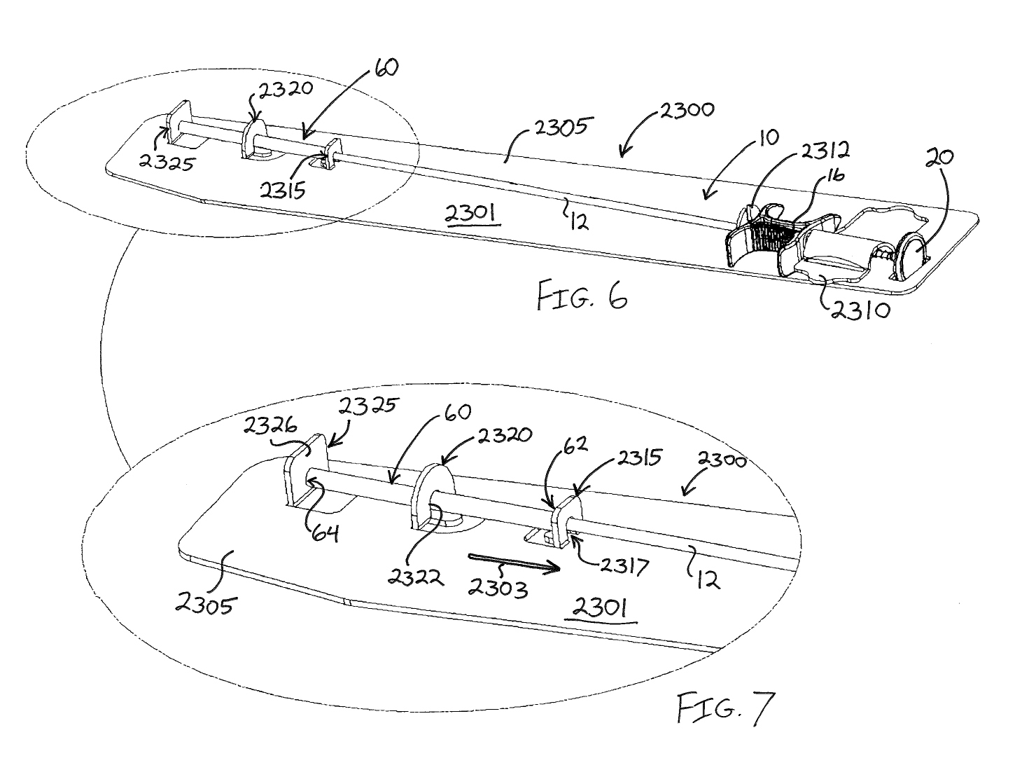

FIG. 4 depicts an exemplary package arrangement for holding marker delivery device (10). The package arrangement is shown including a sterile outer envelope (2100), which includes a bottom portion (2140) and a peelable upper portion (2160). Upper portion (2160) is selectively sealed to bottom portion (2140), such that portions (2140, 2160) maintain the sterility of marker delivery device (10) when portions (2140, 2160) are sealed together. Marker delivery device (10) is shown supported on an internal package member (2300), which is interposed between portions (2140, 2160). Internal package member (2300) of this example will be described in greater detail below, with reference to FIGS. 6-7. In addition, various other exemplary internal package members will be described in greater detail below. It should be understood that the various exemplary internal package members described below may be used to support marker delivery device (10) within an envelope (2100) and/or within a box or other type of container.

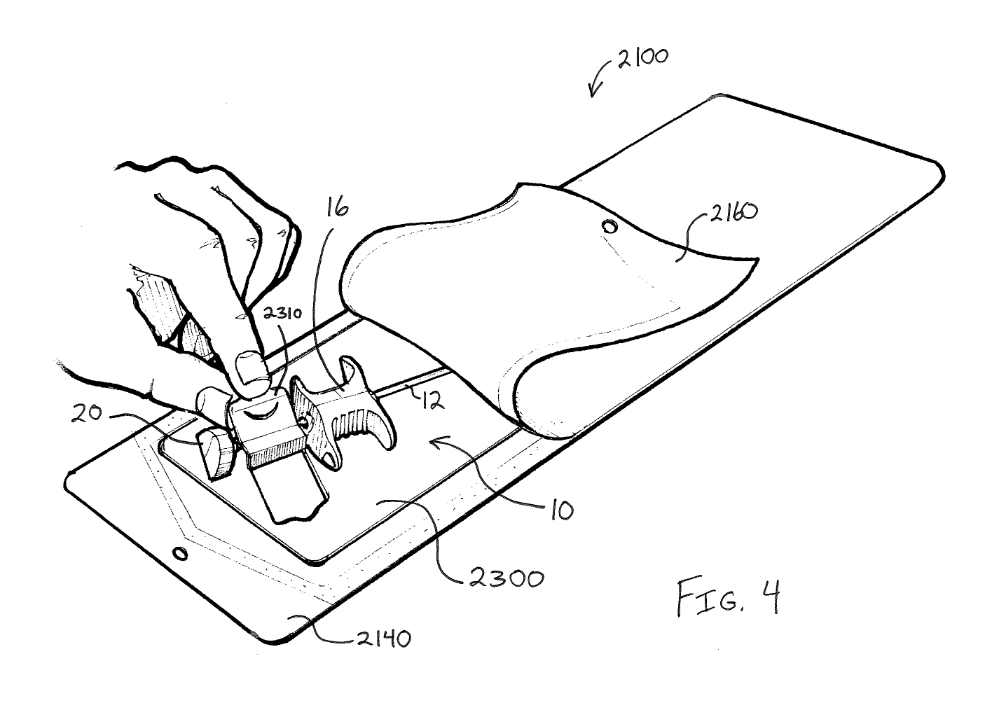

FIG. 5 depicts a conventional internal package member (2200′), which is suitable for holding marker delivery device (10) in an envelope (2100). Internal package member (2200′) includes a proximal tab portion (2210′), an intermediate tab portion (2212′), and a distal tab portion (2220′). Tab portions (2210′, 2212′, 2220′) are integral with a base portion (2205′) and extend from base portion (2205′). Proximal tab portion (2210′) is configured to secure the proximal end of marker delivery device (10) to internal package member (2200′). In particular, proximal tab portion (2210′) is wrapped over push rod (18), between grip (16) and plunger (20), and has its otherwise free end selectively secured to base portion (2205′). Intermediate tab portion (2212′) is configured to abut a distal end of grip (16). Distal tab portion (2220′) is configured to secure a distal portion of marker delivery device (10) to internal package member (2200′). In particular, deployer tube (12) extends through an opening formed in distal tab portion (2200′). Tab portions (2210′, 2212′, 2220′) thus cooperate to substantially secure marker delivery device (10) to internal package member (2200′) and substantially maintain the position of marker delivery device (10) relative to internal package member (2200′).

FIGS. 6-7 illustrate marker delivery device (10) coupled with the exemplary package internal package member (2300) shown in FIG. 4. In particular, FIG. 6 illustrates marker delivery device (10), of the type shown in FIGS. 1-3, having a relatively flexible marker deployer tube (12) as disclosed above. In addition, marker delivery device (10) is shown in FIGS. 6-7 supported by package internal package member (2300) with a sleeve (60) disposed over a distal end of tube (12) to at least partially cover side opening (14). It should be understood that sleeve (60) is just one merely illustrative example of an elongate member that may be used to selectively at least partially cover side opening (14). Various other examples of elongate members will be described in greater detail below; while other examples of suitable elongate members will be apparent to those of ordinary skill in the art in view of the teachings herein. The term “elongate member” should be read to include (but not necessarily be limited to) a member having a length that is at least five times the member's transverse width/diameter.

As shown in FIGS. 6-7 and 10, sleeve (60) of the present example is in the form of tube having an open proximal end (62), an open distal end (64), and an axially extending lumen (66) for receiving flexible deployer tube (12). Sleeve (60) is sized to fit in sliding engagement over the outside diameter of deployer tube (12). In particular, lumen (66) presents an inner diameter that is slightly larger than the outer diameter of deployer tube (12). Furthermore, sleeve (60) is configured to selectively cover side opening (14) while marker delivery device (10) is supported within sterile packaging on package internal package member (2300) (e.g., when sleeve (60) is slid to a distal position). It should be understood that sleeve (60) will substantially prevent the marker/markers inside deployer tube (12) from being inadvertently and/or prematurely deployed from deployer tube (12) when sleeve (60) is positioned over side opening (14). When deployment of one or more markers from deployer tube (12) is desired, sleeve (60) may be slid proximally along deployer tube (12) (e.g., to a proximal position) to at least substantially uncover side opening (14), allowing one or more markers to be deployed through side opening (14).

In addition, sleeve (60) may be relatively more rigid than deployer tube (12), such as by forming sleeve (60) out of a relatively stiffer material than that from which deployer tube (12) if formed, and/or sizing the outer diameter of sleeve (60) to provide a desired ratio of stiffness with respect to tube (12). Accordingly, sleeve (60) may be used to guide and/or steady deployer tube (12) as tip (22) of deployer tube (12) is inserted into a proximal opening in a biopsy device or access cannula, as described in greater detail elsewhere herein. In particular, the presence and properties of sleeve (60) may substantially reduce or prevent buckling or undesired deflection of deployer tube (12) as tip (22) of deployer tube (12) is inserted into a biopsy device or access cannula. In some other versions, sleeve (60) is flexible. In such versions, sleeve (60) may or may not substantially reduce or prevent buckling or undesired deflection of deployer tube (12) as tip (22) of deployer tube (12) is inserted into a biopsy device or access cannula. It should therefore be understood that deployer tube (12) may be more flexible than sleeve (60) in some versions; that sleeve (60) may be more flexible than deployer tube (12) in some versions; and that various relationships as to flexibility may be provided. For instance, sleeve (60) and/or deployer tube (12) may have different sections of different flexibilities, etc.

Package internal package member (2300) may be formed of a relatively thin sheet or sheet like material that can be cut (e.g., die cut, etc.) and folded to provide tabs or other out-of-plane features. For instance, internal package member (2300) may be formed of any suitable medical grade cardboard, plastic, paper stock, or other suitable stock having sufficient rigidity to support marker delivery device (10), and that can have portions cut and folded to form tabs. In FIGS. 6-7, package internal package member (2300) is shown as a unitary structure including a base portion (2305) having an upper surface (2301) and having multiple tab features formed therein. In particular, internal package member (2300) of the present example is shown including a proximal tab (2310) for holding a proximal portion of marker delivery device (10) with respect to internal package member (2300); and a tab (2312) just distal of grip (16). In addition, internal package member (2300) includes a relatively distal tab (2315), a second still more distal tab (2320), and a third distal-most tab (2325). Tabs (2310, 2312, 2315, 2320, 2325) may be formed by cutting and folding spaced apart portions of base (2305) of internal package member (2300). Alternatively, tabs (2310, 2312, 2315, 2320, 2325) may be formed using any other suitable techniques.

Tab (2320) has an arch like shape that partially encircles sleeve (60), with tab (2320) providing an opening (2322). Sleeve (60) extends through opening (2322) of tab (2320) such that proximal end (62) of sleeve (60) is positioned proximal of tab (2320) and distal end (64) of sleeve (60) is positioned distal of tab (2320). Opening (2322) is sized so that sleeve (60) may slide/translate in a proximal direction relative to tab (2320), with tab (2320) restraining sleeve (60) (and thus tube (12)) from moving in a direction perpendicular to the axes of tube (12) and sleeve (60). In other words, tab (2320) acts to restrain vertical movement of sleeve (60) and tube (12) away from base (2305), as well as sideways movement of sleeve (60) and tube (12) relative to base (2305), but not longitudinal/axial movement of sleeve (60) and tube (12) relative to base (2305).

Tab (2325) is disposed distally relative to tab (2320). Tab (2325) is shown having a generally planar configuration extending perpendicular to base (2305). Tab (2325) has a proximally facing surface (2326) that abuts distal end (64) of sleeve (60). Tab (2325) is configured to prevent sleeve (60) from translating/sliding relative to tube (12) and base (2305) in a distal direction (direction parallel to axis of sleeve (60) and tube (12)); but tab (2325) does not prevent sleeve (60) (and tube (12)) from being slid/translated in a proximal direction (direction indicated by arrow (2303) in FIG. 7). Proximally directed arrow (2303) extends in a direction generally parallel to the longitudinal axes of sleeve (60) and tube (12).

Tab (2315) has an arch like shape and provides an opening (2317). Opening (2317) is sized to permit proximal sliding/translation of tube (12) through opening (2317) of tab (2315), while preventing proximal movement of sleeve (60) therethrough. For instance, tab (2315) may be configured such that at least a portion of proximal end (62) of sleeve (60) abuts against a distal facing surface of tab (2315).

Accordingly, tabs (2315, 2320, 2325) assist in restraining movement of the distal portion of marker delivery device (10) in a direction that is transverse to the axis of tube (12) and sleeve (60). In addition, tabs (2315, 2320, 2325) permit proximal sliding of the distal portion of tube (12) in the direction indicated by arrow (2303), while holding sleeve (60) from moving proximally in the direction indicated by arrow (2303) relative to internal package member (2300). Of course, the configuration and arrangement of tabs (2315, 2320, 2325) described herein are merely illustrative examples. Various other suitable ways in which tabs (2315, 2320, 2325) may be configured will be apparent to those of ordinary skill in the art in view of the teachings herein.

Referring back to FIG. 4, marker delivery device (10) and sleeve (60) may be supported on internal package member (2300) and disposed within a package (2100). At the time of use, upper portion/cover (2160) of sterile package (2100) may be peeled back from bottom portion (2140), and the fingers of a user's hand may release proximal tab (2310) from marker delivery device (10) (e.g., by bending tab (2310) away from the proximal portion of marker delivery device (10)). Then, using the same fingers of the same hand, the user may slide/translate marker delivery device (10) parallel to axis of tube (12) (e.g., along direction of arrow (2303) shown in FIG. 7), such that marker delivery device (10) is removed from internal package member (2300) with a single hand, while sleeve (60) is retained between tabs (2315, 2325). Accordingly, side opening (14) of tube (12) remains covered by sleeve (60) while marker delivery device (10) is packaged in package (2100), up until the time of intended use. Then with a single hand, the marker delivery device (10) can be slid from internal package member (2300) (and package (2100)), leaving the sleeve (60) with internal package member (2300), so that no other manipulations and/or the use of another hand is not required to remove sleeve (60) from delivery tube (12). Of course, a user may use two hands to remove marker delivery device (10) from internal package member (2300) if desired.

In some instances, it may desirable to provide a guide when inserting flexible deployment tube (12) into a biopsy device or access cannula. If desired, sleeve (60) may be removed from internal package member (2300). For instance, the user may bend tab (2325) distally to provide clearance for sleeve (60), then slide sleeve (60) distally off of internal package member (2300). Alternatively, the user may bend tab (2315) proximally to provide clearance for sleeve (60), then slide sleeve (60) proximally off of internal package member (2300). As yet another merely illustrative variation, one or more of tabs (2315, 2320, 2325) may include one or more perforations to facilitate ripping of one or more of tabs (2315, 2320, 2325) to facilitate removal of sleeve (60) from internal package member (2300). Other suitable ways in which sleeve (60) may be removed from internal package member (2300) will be apparent to those of ordinary skill in the art in view of the teachings herein. It should also be understood that, while the present example includes removal of marker delivery device (10) from internal package member (2300) separate from removal of sleeve (60) from internal package member (2300), internal package member (2300) may be configured to permit marker delivery device (10) and sleeve (60) to be removed from internal package member (2300) together.

FIGS. 8A-8B illustrate an example of how sleeve (60) may be used as a guide to help stiffen flexible deployment tube (12) and otherwise help to guide and position distal tip (22) into an access cannula (3000). While the example depicted in FIGS. 8A-8B shows sleeve (60) and marker delivery device (10) being used with an access cannula (3000), it should be understood that sleeve (60) and marker delivery device (10) may be used in a similar fashion with a biopsy device or other type of device providing access to a biopsy site. In FIG. 8A, marker deliver device (10) is shown with sleeve (60) positioned over the distal portion of deployer tube (12), such that sleeve (60) covers side opening (14) of deployer tube (12). Sleeve (60) is shown having a finger recess (68) to facilitate gripping. FIG. 8A also depicts an access cannula (3000) positioned through a skin surface (3200) (e.g., a patient's breast). Access cannula (3000) of this example includes a hub portion (3010) having an open proximal end; and an access tube (3020) extending distally from hub (3010). Access tube (3020) has an internal lumen communicating with the open proximal end of hub (3010). The internal lumen of tube (3020) extends to an open distal end (3022) in the present example. Alternatively, an access cannula with a closed distal end and a side opening alignable with side opening (14) could also be used.

Hub portion (3010) may remain outside the skin (3200), and access tube (3020) may extend distally from hub (3010) through the skin surface (3200), with open distal end (3022) of tube (3020) positioned in relation to a biopsy cavity (3250) or other biopsy site. Access tube (3020) may be inserted into a pre-formed biopsy tract; or tube (3020) be inserted/introduced into the tissue with a stylet or obturator (not shown) having a piercing distal end suitable for extending through an internal lumen of tube (3020).

Still referring to FIG. 8A, distal end (64) of sleeve (60) may be sized and/or shaped such that distal end (64) of sleeve (60) abuts, but does not enter, the proximal opening of hub (3010). In other words, with sleeve (60) positioned over the distal portion of the deployer tube (12) (and covering side opening (14)), distal end (64) of sleeve (60) may be positioned against the proximal end of hub (3010) prior to inserting deployer tube (12) into the breast via access tube (3020). If desired, and as will be described in greater detail below, sleeve (60) may include a collar or flange on distal end (64) to prevent sleeve (60) from being over-inserted into hub (3010). In addition or in the alternative, and as will also be described in greater detail below, distal end (64) of sleeve (60) may include a distally projecting portion (e.g., frustoconically shaped) that is at least partially inserted into hub (3010) (e.g., to facilitate alignment of deployer tube (12) axis with access tube (3020) axis, etc.). As yet another merely illustrative variation, the proximal end of hub (3010) may include a recess configured to receive at least part of distal end (64) of sleeve (60) (e.g., to facilitate alignment of deployer tube (12) axis with access tube (3020) axis, etc.).

Referring to FIG. 8B, with one hand holding sleeve (60) stationary with respect to hub (3010), the other hand of the user may be used to advance/slide the entire marker delivery device (10) distally (but without deploying a marker yet), such that deployer tube (12) slides distally through sleeve (60) and then through access tube (3020), so that side opening (14) is eventually positioned beyond the open distal end (3022) of the access tube (3020) to deploy a marker into the biopsy cavity (3250). With side opening (14) positioned beyond the open distal end (3022), pushrod (18) may then be actuated to deploy one or more markers out of side opening (14) and into the biopsy cavity (3250). By providing sleeve (60) in sliding relationship with deployer tube (12), the relatively stiffer sleeve (60) acts to guide and stabilize the relatively more flexible deployer tube (12) as deployer tube (12) is introduced through access cannula (3000). As can be seen, sleeve (60) is at a distal position relative to deployer tube (12) when deployer tube (12) is being positioned for insertion in access cannula (3000) (FIG. 8A); then sleeve (60) is at a proximal position relative to deployer tube (12) when deployer tube (12) has been inserted through access cannula (3000) (FIG. 8B).

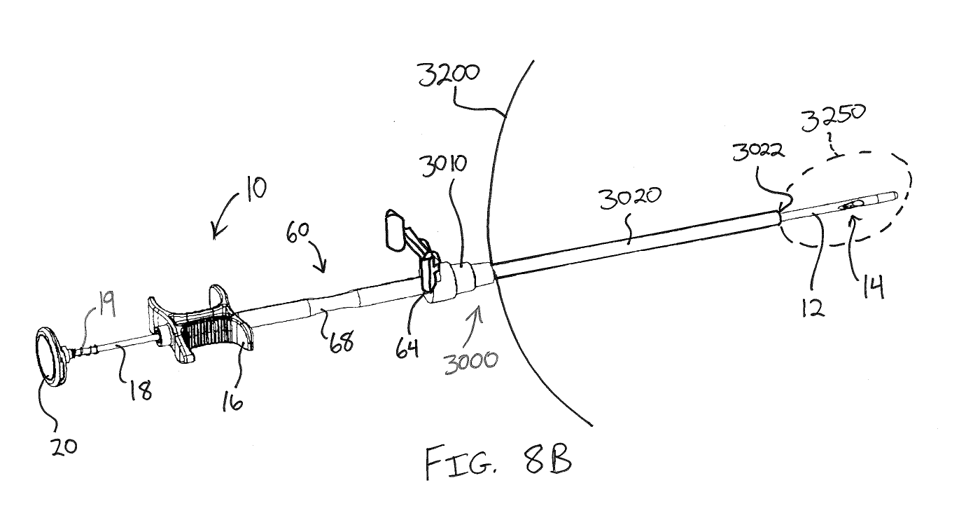

In some versions, as shown in FIG. 9, grip (16) includes an integral distal projection (1601) with an outwardly extending annular flange (1602). Projection (1601) and flange (1602) are configured to enter lumen (66) of sleeve (60) and become lodged therein when sleeve (60) reaches the proximal position shown in FIGS. 8B and 9. In other words, projection (1601) and flange (1602) are configured to substantially secure sleeve (60) to grip (16). Such engagement between flange (1602) and sleeve (60) may be in the form of an interference fit, a snap fit, or some other type of engagement. It should also be understood that the engagement between flange (1602) and sleeve (60) may be configured to permit subsequent disengagement of sleeve (60) from projection (1601) and flange (1602). As with other components described herein, projection (1601) and flange (1602) may be varied, substituted, supplemented, or even omitted as desired.

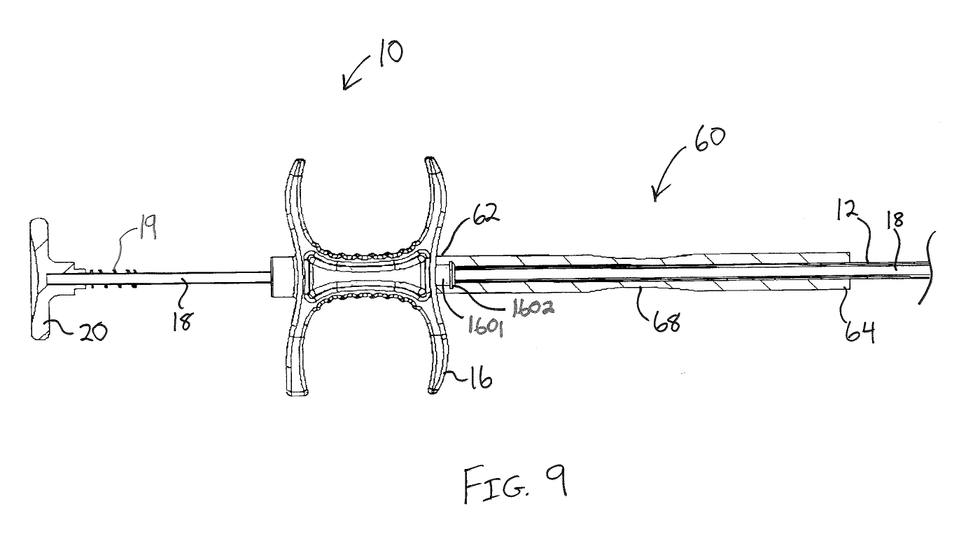

Referring to FIG. 10, the sleeve (60) may have a length (L) less than half the length of flexible deployer tube (12), and a length (L) that is at least twice the axial length of side opening (14). For instance, the length (L) of sleeve (60) may be less than about 1/3 the length of deployer tube (12) (e.g., measured from tip (22) to grip (16)). In addition or in the alternative, the length (L) of sleeve (60) may be at least about four times the length of side opening (14). In some versions, the length (L) is at least about four centimeters, and more particularly, at least about 6 centimeters; and the outer diameter of sleeve (60) is between about 4 mm and about 6 mm, with length (L) being at least about 10 times the outer diameter. The inner diameter of the sleeve (60) (e.g., the diameter of lumen (66), which extends the full length (L) of sleeve (60)) may be slightly larger than the outer diameter of flexible deployer tube (12) to provide for sliding of sleeve (60) axially along at least a part of the length of the flexible deployer tube (12). Of course, any of the foregoing dimensions of sleeve (60) may be varied as desired. Similarly, such dimensions of sleeve (60) may have any other suitable relationships (if any) with dimensions of other components referred to herein.

FIG. 11 illustrates another example of sleeve (60), having a depth stop (69) disposed on the outer surface of sleeve (60). Depth stop (69) may have an outer diameter that is greater than the outer diameter of sleeve (60). Depth stop (69) may be in the form of a flexible o-ring that is sized to fit snugly over the sleeve (60) and slidably positioned at various locations along the sleeve (60), such as by “rolling” the o-ring from one location to another. The outer diameter of sleeve (60) in the example of FIG. 11 may be sized to fit at least partially within an opening in an access cannula or biopsy device. Depth stop (69) may be positioned at a desired position on sleeve (60), such that only a predetermined portion of the length of sleeve (60) (e.g. the portion between stop (69) and end (64)) is able to enter an access cannula or biopsy device opening, with depth stop (69) providing a restriction on the depth to which sleeve (60) may be inserted in an access cannula or biopsy device, etc.

If desired, sleeve (60) may include markings (69A) corresponding to various insertion depths, and stop (69) may be positioned along sleeve (60) using markings (69A). The example shown in FIG. 11 may be used with different flexible deployer tubes (12) and/or different length access cannulas and/or different length biopsy needles, with stop (69) being positionable along the sleeve (60) to provide a desired location of the side opening (14) with respect to the biopsy cavity. Referring back to FIG. 8A, flexible deployer tube (12) may also include markings (12A), which may be employed with respect to depth of insertion of flexible tube (12) into sleeve (60) and/or to align side opening (14) with respect to a desired angular/o'clock position.

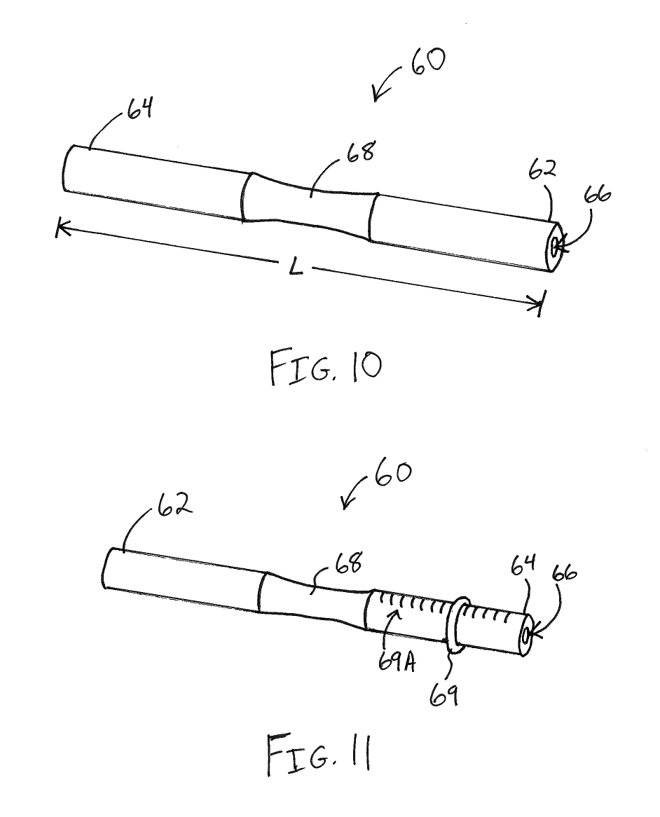

FIG. 12 shows another exemplary sleeve (160). Sleeve (160) of this example includes an open proximal end (162), an open distal end (164), and an axially extending lumen (166) for receiving flexible deployer tube (12). Like sleeve (60), sleeve (160) is sized to fit in sliding engagement over the outside diameter of deployer tube (12). Sleeve (160) also includes a finger recess (168) to facilitate gripping of sleeve (160). However, unlike sleeve (60), sleeve (160) of this example includes threading (170) between finger recess (168) and distal end (164). A nut (172) is disposed about threading (170), and includes internal threading (not shown) that meshes with threading (170). The longitudinal position of nut (172) along sleeve (160) is thus adjustable by selectively rotating nut (172) relative to sleeve (160) in either direction. While nut (172) is shown as having a hexagonal profile, it should be understood that nut (172) may have any other suitable configuration (e.g., round with knurled outer surface, etc.).

In this example, nut (172) is configured to act as a depth stop, similar to depth stop (69) described above, to selectively restrict the depth to which distal end (164) of sleeve (160) may be inserted into an access device such as hub (3010) of access cannula (3000), a biopsy device, probe, or any other type of device providing access to a biopsy site or other type of site within a patient. In other words, in some exemplary uses, an access device presents an inner diameter that is greater than the outer diameter of distal end (164) yet less than the outer diameter presented by nut (172), such that distal end (164) may be inserted into the lumen of the access device until nut (172) abuts some surface or feature of the access device. It should be understood that the depth to which sleeve (160) may be inserted in an access device may influence the depth to which marker delivery device (10) may be inserted in the access device. For instance, referring back to FIG. 8B, it may be seen that sleeve (60) itself acts as a depth stop by simultaneously abutting grip (16) and hub (3010). In other words, the length of sleeve (60) governs the depth to which marker delivery device (10) may be inserted in access cannula (3000). Referring back to FIG. 12, it should be understood that, with at least part of distal end (164) being insertable into an access device until nut (172) reaches the access device, adjusting the longitudinal position of nut (172) along sleeve (160) may essentially adjust the effective length of sleeve (160), thereby adjusting the depth to which marker delivery device (10) may be inserted in the access device. Such adjustability of depth of insertion may ultimately provide variable positions at which one or more markers may be deployed through side opening (14) of deployer tube (12). It should also be understood that nut (172) may be self-adjusting.

It should be understood from the foregoing that depth stop (69) and nut (172) may provide similar functionalities. Among those functionalities described above (namely, serving as a depth stop), depth stop (69) and nut (172) may also provide a barrier to separate a user's fingers and/or thumb from contacting sleeve (60, 160), an access device (e.g., access cannula (3000), etc.), and/or deployer tube (12). In addition, depth stop (69) and nut (172) may facilitate gripping of sleeve (60, 160) and provide a leverage point to push and/or pull sleeve (60, 160) along deployer tube (12). Other functionalities that may be provided by depth stop (69) and/or nut (172) will be apparent to those of ordinary skill in the art in view of the teachings herein.

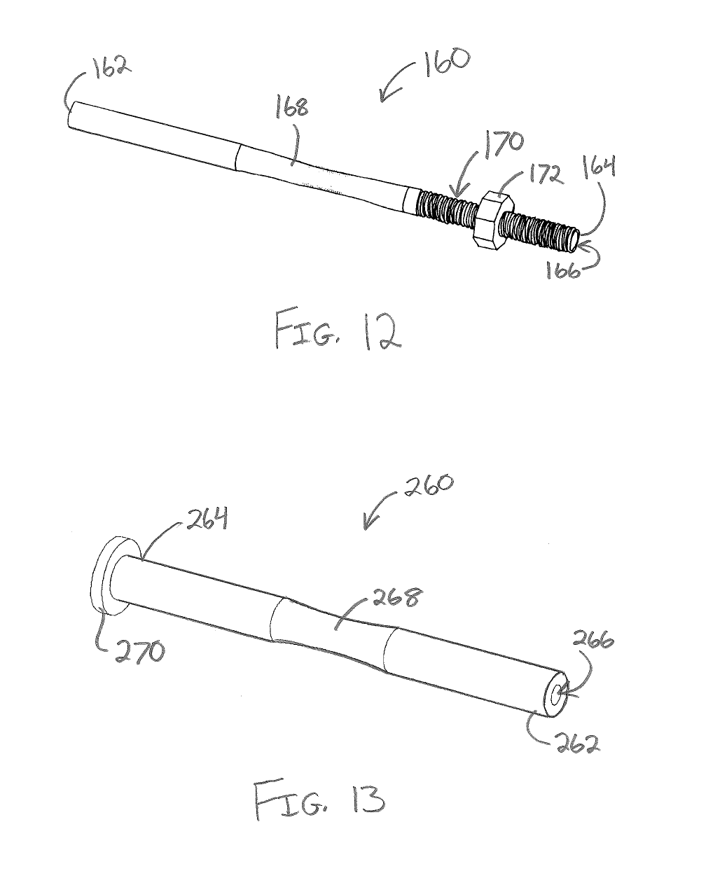

FIGS. 13-14 show another exemplary sleeve (260). Sleeve (260) of this example includes an open proximal end (262), an open distal end (264), and an axially extending lumen (266) for receiving flexible deployer tube (12). Like sleeves (60, 160), sleeve (260) is sized to fit in sliding engagement over the outside diameter of deployer tube (12). Sleeve (260) also includes a finger recess (268) to facilitate gripping of sleeve (260). In addition, sleeve (260) includes an integral flange (270) at distal end (264). While flange (270) is located at distal end (264) in the present example, it should be understood that flange (270) may be located elsewhere along sleeve (260) (e.g., at proximal end (262), anywhere between ends (262, 264), etc.). It should also be understood that sleeve (260) may include any suitable number of flanges (270) at various locations. Like depth stop (69) and nut (172), flange (270) may prevent sleeve (260) from being over-inserted in an access cannula (3000), biopsy device, probe, or other type of access device. Also like depths stop (69) and nut (172), flange (270) may provide a barrier to separate a user's fingers and/or thumb from contacting sleeve (270), an access device (e.g., access cannula (3000), etc.), and/or deployer tube (12). In addition, flange (270) may facilitate gripping of sleeve (260) and provide a leverage point to push and/or pull sleeve (260) along deployer tube (12). Other functionalities that may be provided by flange (270) will be apparent to those of ordinary skill in the art in view of the teachings herein.

As best seen in FIG. 14, sleeve (260) also includes a frustoconical projection (272) protruding from distal end (264), distal to flange (270). Projection (272) is configured to fit in the lumen of hub (3010), and the frustonical configuration of projection (272) is configured to help guide projection (272) into the lumen of hub (3010). Projection (272) also includes a notch (274) that is configured to receive a complementary feature of hub (3010). Of course, as with other components described herein, notch (274) is merely optional. Furthermore, projection (272) is merely optional, and may be configured in any other suitable fashion or may even be omitted altogether if desired.

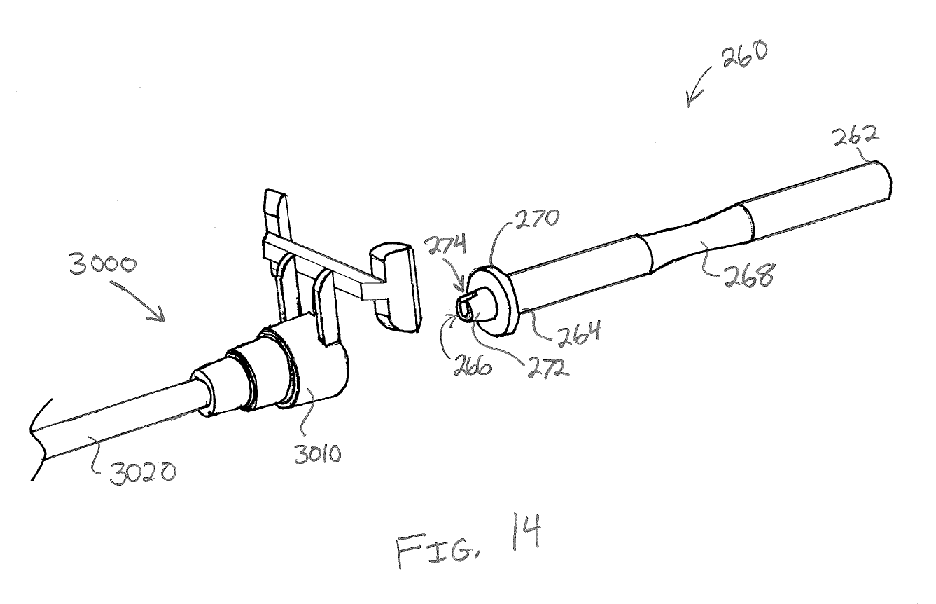

FIGS. 10-14 illustrate sleeves (60, 160, 260) having a closed cross-section shape. FIG. 15 illustrates another exemplary sleeve (70) having a body (75) with an open, generally C-shaped cross section. Sleeve (70) of this example has a recess or opening (76) that extends from a proximal end (72) to a distal end (74). Opening (76) is sized so that the sleeve (70) may be snapped over flexible deployer tube (12) (e.g., as opposed to inserting tip (22) through proximal end (72) of the sleeve (70), though such a technique may be used if desired). Sleeve (70) may be employed to selectively cover side opening (14) prior to use of marker delivery device (10). Sleeve (70) may also be employed as a guide (e.g., in the manner described above with respect to sleeve (60), etc.) to position the side opening (14) and support/stabilize flexible deployer tube (12) as shown in FIGS. 8A-8B. It should therefore be understood that sleeve (70) may be slidable along at least part of the length of deployer tube (12), much like sleeve (60) described above. Furthermore, sleeve (70) may be formed of a resilient material, such that sleeve (70) may be selectively snapped onto and off of deployer tube (12), such as by moving sleeve (70) relative to deployer tube (12) along a path that is substantially transverse to the longitudinal axis defined by deployer tube (12) and/or along any other suitable path.

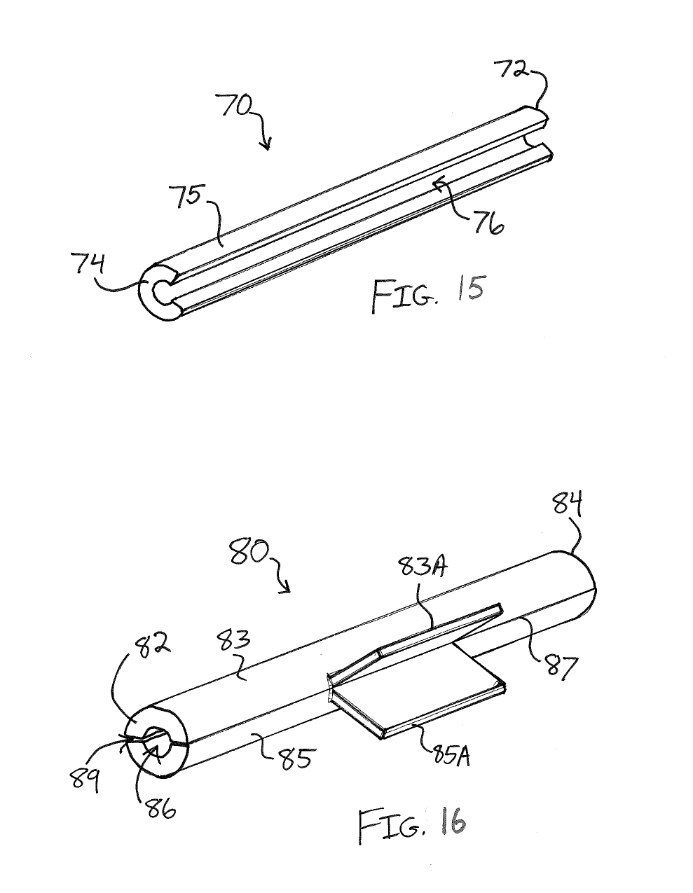

FIG. 16 illustrates a perspective view of another exemplary sleeve (80) suitable for selectively covering side opening (14) and/or guiding flexible deployer tube (12). Sleeve (80) in this example is provided in the form of a clip having a first portion (83) that is resiliently biased toward a second portion (85), such as with a spring or living hinge (87). First portion (83) and second portion (85) each have generally semicircular cross-sections, which when biased toward one another (as shown in FIG. 16) provide a lumen (86) extending from a proximal end (82) to a distal end (84). Lumen (86) is sized for receiving flexible deployer tube (12). Sleeve (80) also includes tabs (83A, 85A), which may be pressed together to cause portions (83, 85) to separate along a split line (89) to receive deployer tube (12). When tabs (83A, 85A) are released, the resilient bias at living hinge (87) urges first and second portions (83, 85) toward each other to a closed position. It should therefore be understood that tabs (83A, 85A) may be selectively pinched toward each other and released to selectively unsecure and secure sleeve (80) relative to deployer tube (12) with a transverse movement of sleeve (80) relative to deployer tube (12). Sleeve (80) may still be configured to translate relative to deployer tube (12) when sleeve (80) is disposed about deployer tube (12). It should be understood that sleeve (80) may cover side opening (14) of deployer tube (12); and may be used in the manner described above with respect to sleeve (60) to guide/support flexible deployer tube (12) as it is inserted into an access cannula or biopsy device, etc.

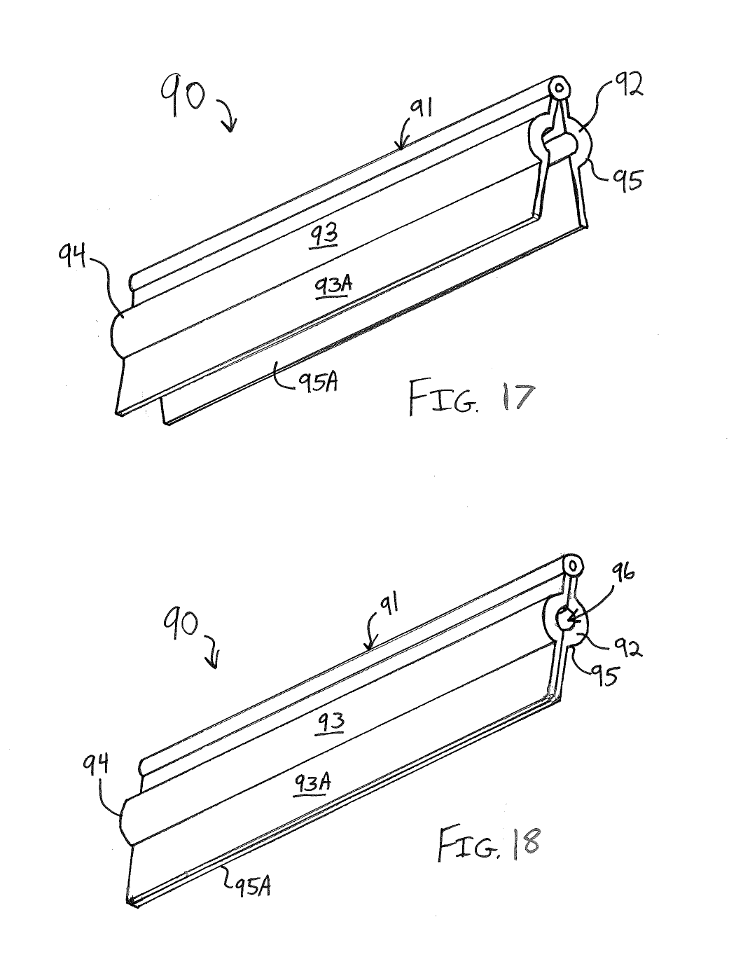

FIGS. 17-18 illustrate perspective views of yet another example of a sleeve (90) that may be used to selectively cover side opening (14) and/or to guide/support the tube (12). FIG. 17 shows sleeve (90) in an open configuration and FIG. 18 shows sleeve (90) in a closed configuration. Sleeve (90) of this example includes first and second portions (93, 95) joined by a hinge (91). Hinge (91) may be resiliently biased to urge portions (93, 95) to the closed configuration. Alternatively, hinge (91) may be substantially malleable. Tabs (93A, 95A) are illustrated for providing leverage to close portions (93, 95) about deployer tube (12). Portions (93, 95), when in the closed configuration, provide a lumen (96) extending from a proximal end (92) to a distal end (94). When portions (93, 95) are in an open configuration, sleeve (90) may laterally receive deployer tube (12), such that portions (93, 95) may thereafter be closed about deployer tube (12) with deployer tube (12) being positioned in lumen (96). Sleeve (90) may still be configured to translate relative to deployer tube (12) when sleeve (90) is disposed about deployer tube (12). It should be understood that sleeve (90) may be used to cover side opening (14) of deployer tube (12); and may be used in the manner described above with respect to sleeve (60) to guide/support flexible deployer tube (12) as it is inserted into an access cannula or biopsy device, etc.

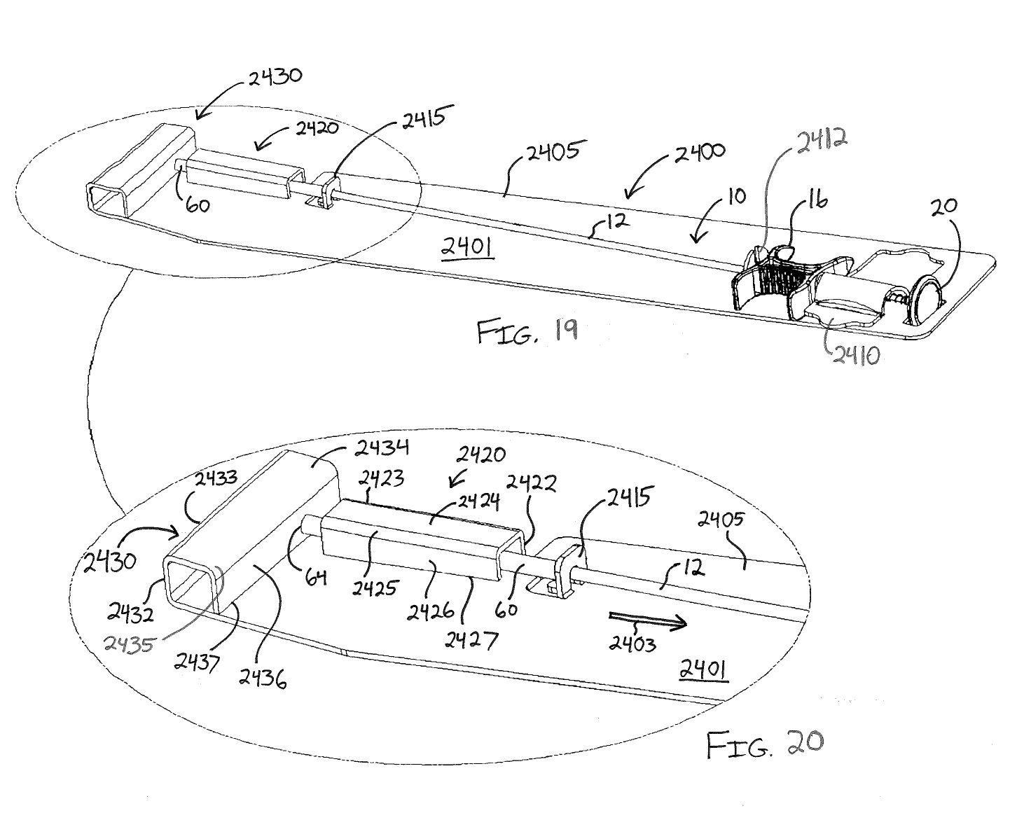

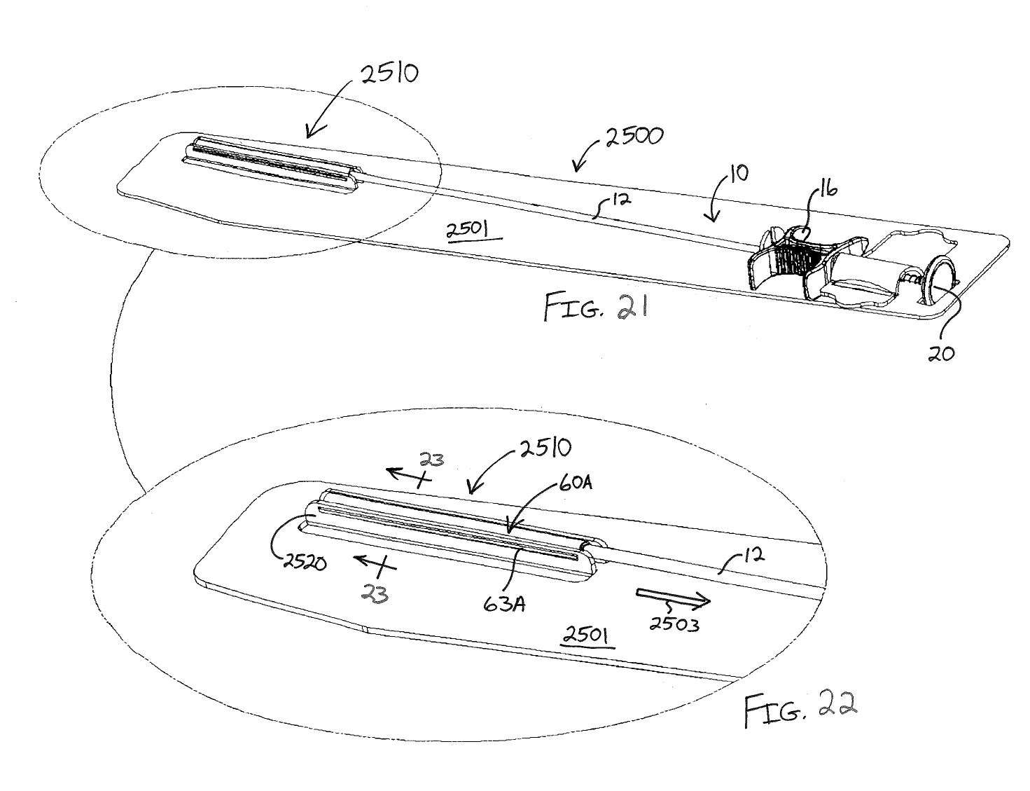

FIGS. 19-20 show another exemplary package internal package member (2400) for supporting a biopsy marker delivery device (10) with a sleeve (60). While sleeve (60) is shown in FIGS. 19-20, it should be understood that internal package member (2400) may also be used with any other sleeve (70, 80, 90, etc.) disposed about deployer tube (12). Internal package member (2400) of this example includes a base portion (2405) having an upper surface (2401). Internal package member (2400) also includes tabs (2410, 2412, 2415) that are substantially similar to tabs (2310, 2312, 2315), respectively, shown in of FIG. 6 and described above. For instance, tab (2410) is configured to selectively hold a proximal portion of marker delivery device (10) relative to internal package member (2400). Tab (2412) substantially restricts distal movement of grip (16) relative to internal package member (2400). Tab (2415) permits proximal sliding of deployer tube (12) but prevents proximal sliding/translation of sleeve (60) along the direction of the longitudinal axis of sleeve (60) and deployer tube (12).

In addition, internal package member (2400) includes a more distal tab structure (2420) and a distal most tab structure (2430). As best seen in FIG. 20, tab structure (2420) includes side panels (2422, 2426) and a top panel (2424). Panels (2422, 2424, 2426) enclose a portion of the length of sleeve (60) and hold sleeve (60) against surface (2401) in this example. In some versions, panels (2422, 2424, 2426) simply enclose a portion of the length of sleeve (60) and do not hold sleeve (60) against surface (2401). Fold (2423) separates panels (2422, 2424); while fold (2425) separates panels (2424, 2426). The free end (2427) of panel (2426) may be inserted in a slot (not shown) in base (2405). Alternatively, free end (2427) may be dealt with in any other suitable fashion.

Tab structure (2430) includes panels (2432, 2434, 2436). Panel (2436) abuts distal end (64) of sleeve (60) to prevent distal sliding/translation of sleeve (60). Panels (2432, 2434) are joined at fold (2433); while panels (2434, 2436) are joined at fold (2435). The free end (2437) of panel (2436) may be inserted into a slot (not shown) in base (2405). Alternatively, free end (2437) may be dealt with in any other suitable fashion. As with other tabs described herein, tabs (2410, 2412, 2415, 2420, 2430) are formed by making cuts (e.g., through die cutting) in base (2405) and then folding cut portions relative to base (2405) such that the cut portions protrude above top portion (2410) of base (2405) to form tabs (2410, 2412, 2415, 2420, 2430). Various other suitable ways in which tabs (2410, 2412, 2415, 2420, 2430) may be formed and configured will be apparent to those of ordinary skill in the art in view of the teachings herein.

Prior to use of marker delivery device (10), sleeve (60) covers side opening (14) of deployer tube (12). With internal package member (2400) disposed within a partially opened sterile envelope, marker delivery device (10) may be slid proximally along the axis of deployer tube (12) (in the direction of arrow (2403)) to remove deployer tube (12) through the opening in tab (2415), while leaving sleeve (60) captured between tab (2415) and tab panel (2436). If it is desired to use sleeve (60) in a manner similar to that shown in FIGS. 8A-8B, internal package member (2400) and sleeve (60) may be removed together from the sterile packaging, and then tab structure (2420) may be unfolded way from sleeve (60), and sleeve (60) may be lifted free (e.g., in an upward direction) from base (2405). Tip (22) of deployer tube (12) may then be inserted into lumen (66) of sleeve (60), and distal end (64) may be positioned at hub (3010) of access cannula (3000), at an entry point in a biopsy device, or elsewhere, such that sleeve (60) may be used to guide further distal insertion of deployer tube (12).

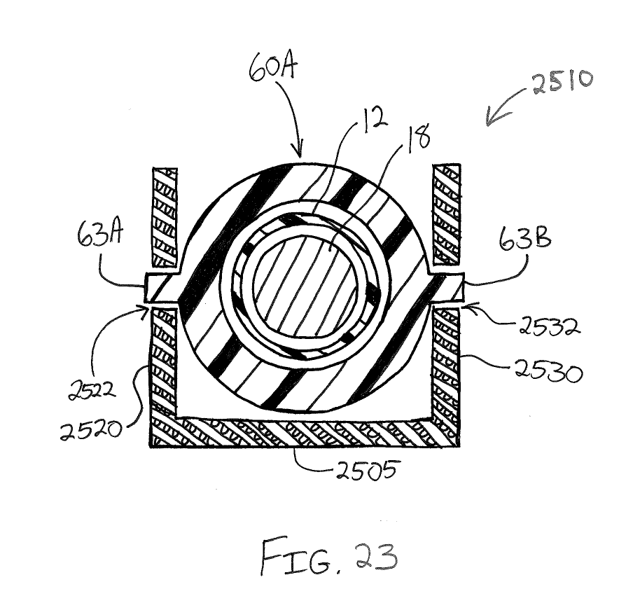

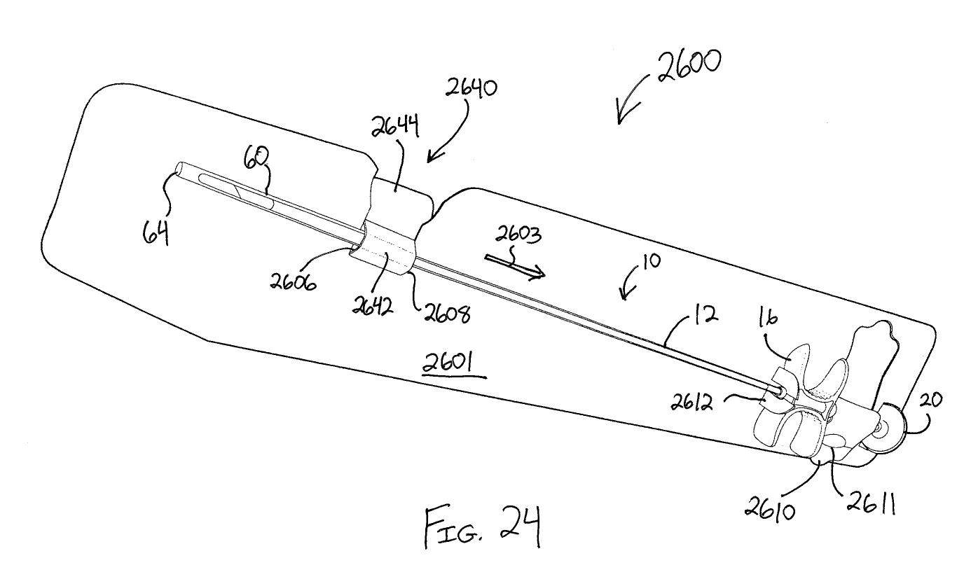

FIGS. 21-23 depict another exemplary package internal package member (2500) for supporting a marker delivery device (10) with a sleeve (60A). Internal package member (2500) of this example is substantially similar to internal package members (2300, 2400) described above, except that internal package member (2500) includes a tab assembly (2510). Tab assembly (2510) extends along at least a portion of the length of sleeve (60A), which is disposed in sliding relationship over the distal portion of deployer tube (12). As best seen in FIG. 23, sleeve (60A) of this example includes outwardly extending members (63A, 63B) that extend like rails along the length of sleeve (60A) and parallel to the longitudinal axis of sleeve (60A). Sleeve (60A) in this example is otherwise configured the same as sleeve (60) described above. Of course, sleeve (60A) may have any other suitable configuration, including but not limited to variations of outwardly extending members (63A, 63B).

Tab assembly (2510) includes a first tab (2520) and a second tab (2530). Tabs (2520, 2530) extend upwardly relative to top surface (2501) and are substantially parallel to each other. Tab (2520) includes an elongated slot (2522) that is sized to receive outwardly extending member (63A); and tab (2530) includes an elongated slot (2532) sized to receive outwardly extending member (63B), as shown in FIG. 23. Outwardly extending members (63A, 63B) engage slots (2522, 2532) in tabs (2520, 2530), thereby substantially holding sleeve (60A) in place on internal package member (2500). Tabs (2520, 2530) may be deflected outwardly to disengage outwardly extending members (63A, 63B) from slots (2522, 2532), thereby disengaging sleeve (60A) from internal package member (2500).

When it is desired to remove marker delivery device (10) from internal package member (2500) with internal package member (2500) in the partially opened sterile outer packaging (2100) (see FIG. 4), grip (16) may be grasped by a single hand to slide/translate marker delivery device (10) (including deployer tube (12)) out of sterile outer packaging (2100) in the direction indicated by arrow (2503) until deployer tube (12) is clear of sleeve (60A). Once marker delivery device (10) is removed from internal package member (2500) (and outer packaging (2100)), internal package member (2500) may be removed from outer packaging (2100), if desired; and sleeve (60A) may be removed from internal package member (2500) by folding back tabs (2520, 2530) toward top surface (2501) to disengage outwardly extending members (63A, 63B) from elongated slots (2522, 2523). Sleeve (60A) and marker delivery device (10) may then be used in accordance with any of the teachings herein; or as otherwise desired. As another merely illustrative example, tabs (2520, 2530) may be folded toward top surface (2501) to disengage outwardly extending members (63A, 63B) from elongated slots (2522, 2523) while sleeve (60A) is still disposed on deployer tube (12), such that sleeve (60A) and marker delivery device (10) may be removed together as an assembly from internal package member (2500).

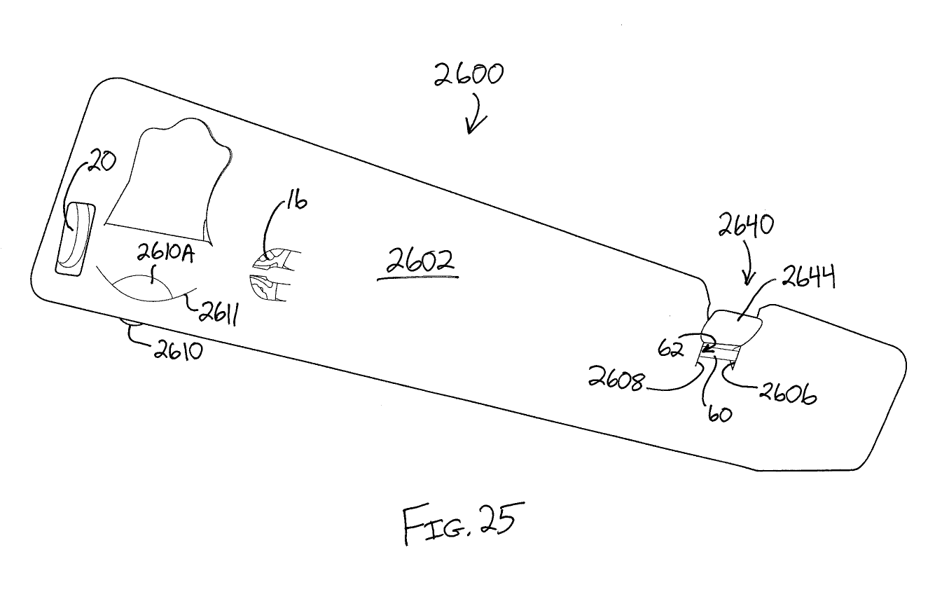

FIGS. 24-25 show top and bottom views of yet another exemplary package internal package member (2600) suitable for holding marker delivery device (10) and sleeve (60). Package internal package member (2600) of this example includes a base 2605 having a top surface (2601) and a bottom surface (2602). A proximal tab (2610) is folded over a portion of push rod (18) and holds push rod (18) in a “pre marker delivery position.” As best seen in FIG. 25, tab (2610) includes a free end (2610A) that inserts in a slot (2611) of base (2605). Internal package member (2600) also includes a tab (2612) positioned distal to and abutting grip (16). Additionally, internal package member (2600) includes a distal tab (2640) that is folded over a proximal portion of sleeve (60). Distal tab (2640) is defined, at last in part, by cut edges (2608, 2606) of base (2505). An intermediate portion (2642) of tab (2640) extends over sleeve (60); and a free end portion (2644) of tab (2640) is tucked under edges (2606, 2608) to engage bottom surface (2602).

Tab portion (2642) serves to selectively hold sleeve (60) in place on internal package member (2600) while permitting deployer tube (12) to be slid/translated in a proximal direction indicated by arrow (2603) in FIG. 24. In addition, cut edge (2608) is in abutting engagement with a bottom portion of proximal end (62) of sleeve (60) as shown in FIG. 25. Accordingly, edge (2608) provides an axial restraint against proximal end (62) of the sleeve (60), providing further restraint of proximal motion of sleeve (60) as deployer tube (12) is withdrawn from under tab portion (2642). To remove marker delivery device (10) from internal package member (2600), a user may grasp proximal tab (2610) and pull upward away from top surface (2601), freeing free end (2610A) from slot (2611) of base (2605). The user may then pull marker delivery device (10) proximally relative to internal package member (2600), in the direction indicated by arrow (2603) in FIG. 21. In the present example, sleeve (60) will still be engaged with internal package member (2600) at this stage. To free sleeve (60) from internal package member (2600), the user may grasp free end portion (2644) of tab (2640) and pull upward away from top surface (2601), freeing free end portion (2644) from edges (2606, 2608). The user may then pull sleeve (60) upwardly and away from internal package member (2600). Sleeve (60) may then be used with marker delivery device (10) in accordance with the teachings herein; or in any other suitable fashion.

As another merely illustrative example, a user may grasp proximal tab (2610) and pull upward away from top surface (2601), freeing free end (2610A) from slot (2611) of base (2605); then grasp proximal tab (2610) and pull upward away from top surface (2601), freeing free end (2610A) from slot (2611) of base (2605). The user may then simply lift marker delivery device (10) away from internal package member (2600) with sleeve (60) still disposed on deployer tube (12). In other words, sleeve (60) and marker delivery device (10) may be removed together as an assembly from internal package member (2500).

The foregoing examples are provided in the context of a biopsy marker delivery device. However, it will be apparent to those of ordinary skill in the art that the teachings herein may be readily applied in devices useful with radioisotope applications, as in PEM, BSGI, and other imaging methods that may employ a radioisotope or other radiation source in connection with imaging a biopsy procedure.

While several devices and components thereof have been discussed in detail above, it should be understood that the components, features, configurations, and methods of using the devices discussed are not limited to the contexts provided above. In particular, components, features, configurations, and methods of use described in the context of one of the devices may be incorporated into any of the other devices. Furthermore, not limited to the further description provided below, additional and alternative suitable components, features, configurations, and methods of using the devices, as well as various ways in which the teachings herein may be combined and interchanged, will be apparent to those of ordinary skill in the art in view of the teachings herein.

Versions of the devices described above may be actuated mechanically or electromechanically (e.g., using one or more electrical motors, solenoids, etc.). However, other actuation modes may be suitable as well including but not limited to pneumatic and/or hydraulic actuation, etc. Various suitable ways in which such alternative forms of actuation may be provided in a device as described above will be apparent to those of ordinary skill in the art in view of the teachings herein.

Versions of the devices described above may have various types of construction. By way of example only, any of the devices described herein, or components thereof, may be constructed from suitable metals, ceramics, plastics, or combinations thereof. Furthermore, although not required, the construction of devices described herein may be configured to be compatible with or optimize their use with various imaging technologies. For instance, a device configured for use with MRI may be constructed from all non-ferromagnetic materials. Also for instance, when using optional imaging technologies with devices described herein, certain configurations may include modifications to materials of construction such that portions or the device may readily appear in a resultant image. Various suitable ways in which these and other modifications to the construction of devices described herein may be carried out will be apparent to those of ordinary skill in the art in view of the teachings herein.

Versions of the devices described above may have application in conventional medical treatments and procedures conducted by a medical professional, as well as application in robotic-assisted medical treatments and procedures.

Versions of described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A marker delivery device, comprising:
(a) a marker deployer tube having a closed distal end and a marker side exit;
(b) at least one biopsy site marker disposed in the deployer tube; and
(c) an elongate member disposed over a distal portion of the deployer tube, the elongate member at least partially covering the marker side exit, wherein the elongate member has an open distal end and an open proximal end.

2. The marker delivery device of claim 1, wherein the elongate member is slidable from a first position where the elongate member covers the marker side exit to a second position where the elongate member is disposed proximal of the side exit.

3. The marker delivery device of claim 1, wherein the marker deployer tube is more flexible than the elongate member.

4. The marker delivery device of claim 1, wherein marker deployer tube has an axial length, wherein the side exit port has an axial length, and wherein the elongate member has an axial length at least twice the axial length of the side exit port.

5. The marker delivery device of claim 4, wherein the elongate member has an axial length less than ⅓ the axial length of the deployer tube.

6. The marker deliver device of claim 1, wherein the elongate member has a closed transverse cross-section.

7. The marker delivery device of claim 1, wherein the elongate member comprises a hinge along its length.

8. The marker delivery device of claim 1, wherein the elongate member comprises a sleeve having a length of at least 4 centimeters.

9. The marker delivery device of claim 1, wherein the elongate member comprises an elongate body and a depth stop extending outwardly relative to the elongate body.

10. The marker delivery device of claim 9, wherein the depth stop is movable along at least part of the length of the elongate body to selectively vary a distance between the depth stop and an end of the elongate body.

11. An assembly, comprising:
(a) an outer package;
(b) a internal package member disposed within the outer package;
(c) a biopsy marker delivery device releasably held on the internal package member and removable from the internal package member, the biopsy marker delivery device comprising a marker deployer tube having a marker exit; and
(d) an elongate member having an open proximal end and an open distal end, wherein the elongate member is slidably disposed on the marker deployer tube to cover the marker exit.

12. The assembly of claim 11, wherein the internal package member is configured to hold the elongate member in place on the internal package member when the biopsy marker delivery device is removed from the internal package member.

13. The assembly of claim 12, wherein the internal package member comprises at least one internal package member feature configured to restrict proximal axial movement of the elongate member while permitting proximal axial movement of the marker deployer tube.

14. The assembly of claim 13, wherein the marker delivery device is removable from the outer package and the internal package member along a direction generally aligned with the axis of the marker deployer tube.

15. The assembly of claim 11, wherein the internal package member comprises at least two tabs proximal of the proximal end of the elongate member.

16. The assembly of claim 11, wherein the internal package member is configured to permit the biopsy marker delivery device and the elongate member to be removed together, as an assembly, from the internal package member.

17. A method of inserting a biopsy marker delivery device into an access channel, wherein the biopsy marker delivery device comprises a marker deployer tube having a marker exit port, the method comprising the steps of:
(a) removing the biopsy marker delivery device and an elongate member from a package member, wherein the elongate member has an open proximal end and an open distal end, wherein the elongate member is slidable over a distal portion of the marker deployer tube;
(b) holding the elongate member with respect to an entrance to an access channel; and
(c) sliding the marker deployer tube distally relative to the elongate member to position the exit port of the marker deployer tube distally beyond the distal end of the elongate member.

18. The method of claim 17, wherein the act of sliding the marker deployer tube distally further comprises positioning the marker exit port distally beyond the access channel.

19. The method of claim 17, wherein the access channel is defined by part of an access cannula or by part of a biopsy device.

20. The method of claim 17, wherein the elongate member is disposed about the marker deployer tube during the act of removing the biopsy marker delivery device and the elongate member from the package member, such that the biopsy marker delivery device and the elongate member are removed from the package member as an assembly.
